(19) United States
(12) Patent Application Publication
(10) Pub. No.: US 2019/0254647 A1
(43) Pub. Date: Aug. 22, 2019

(54) SPECIMEN RETRIEVAL DEVICE

(71) Applicant: COVIDIEN LP, MANSFIELD, MA (US)

(72) Inventor: SCOTT J. PRIOR, SHELTON, CT (US)

(21) Appl. No.: 16/280,155

(22) Filed: Feb. 20, 2019

Related U.S. Application Data
(60) Provisional application No. 62/633,419, filed on Feb. 21, 2018.

(51) Int. Cl. A61B 17/00; A61B 17/42

(52) U.S. Cl. CPC A61B 17/00234; A61B 17/29; A61B 2017/00287; A61B 17/42

(57) ABSTRACT

A specimen retrieval device for extraction of tissue includes two ports and a specimen receptacle affixed to a support assembly at a distal portion of an inner shaft assembly that passes through the two ports. The first port has a larger diameter than the second port, so that introduction of the second port into the longitudinal bore of the first port introduces the specimen receptacle into a patient's body. In embodiments, the specimen receptacle has two openings which may be used to transfer a tissue specimen from within a patient's body cavity to outside of the patient's body.

### CROSS REFERENCE TO RELATED APPLICATION

The present application claims the benefit of and priority to U.S. Provisional Application Ser. No. 62/633,419, filed on Feb. 21, 2018, the entire contents of which are incorporated herein by reference.

### TECHNICAL FIELD

The present disclosure relates to a specimen retrieval device and, more particularly, to a surgical retrieval device for removing tissue specimens from an internal body cavity.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of a body cavity. The surgeon can then perform diagnostic and/or therapeutic procedures at the surgical site with the aid of specialized instrumentation, such as grasping devices including a forceps or a tenaculum, cutters, applicators, and the like, which are designed to fit through additional cannulas.

When removing certain tissues from the body cavity, for example tumor tissue, it is important that the tumor tissue does not come into contact with healthy or uninvolved tissue. If tumor tissue or tissue parts have to be removed, they may be introduced into a “containment bag,” also referred to herein as a “specimen receptacle” or a “specimen bag,” at the site where the tumor or diseased tissue has been detached from the surrounding tissue, after which the specimen receptacle is withdrawn from the body, thereby minimizing contact of the diseased tissue with healthy tissue.

Improved specimen retrieval devices for use in minimally invasive surgical procedures remain desirable.

### SUMMARY

The present disclosure is directed to surgical apparatuses and kits for use in minimally invasive surgery. Methods for using these surgical apparatuses and kits are also provided.

In embodiments, the present disclosure provides a specimen retrieval device including a first port defining a first longitudinal bore and having a proximal portion and a distal portion, the first longitudinal bore having a first diameter; a second port defining a second longitudinal bore and having a proximal portion and a distal portion, the second longitudinal bore having a second diameter smaller than the first diameter; an inner shaft assembly for passage through the first and second longitudinal bores, the inner shaft assembly having a proximal portion and a distal portion, the distal portion of the inner shaft assembly having a support assembly affixed thereto; and a specimen receptacle supported on the support assembly, the specimen receptacle including a body defining a first opening that may be opened and closed and a second opening spaced from the first opening.

In embodiments, the proximal portion of the first port includes a flange, the flange being dimensioned to prevent passage of the first port into a patient's body cavity. In other embodiments, the proximal portion of the second port includes a flange, the flange being dimensioned to prevent passage of the second port completely into the first longitudinal bore.

In some embodiments, the support assembly includes a pair of resilient fingers which support the specimen receptacle, the resilient fingers being movable from a deformed position within the longitudinal bore of the second port to a non-deformed state to open a first mouth of the specimen receptacle to form the first opening in a deployed state.

Kits of the present disclosure include the specimen retrieval device described herein in combination with at least one additional component such as cannulas, grasping devices, or combinations thereof.

Methods of the present disclosure include introducing a first port of a specimen retrieval device through a body opening, the first port defining a longitudinal bore of a first diameter and having a proximal portion and a distal portion, and introducing a second port into the longitudinal bore of the first port, the second port defining a longitudinal bore of a second diameter and having a proximal portion and a distal portion, the second diameter being smaller than the first diameter. An inner shaft assembly is introduced into the longitudinal bores of the first and second ports to position a specimen receptacle supported on a support assembly on the distal portion of the inner shaft within the longitudinal bores of the first and second ports, the inner shaft assembly having a proximal portion and a distal portion. The second port is passed distally through the longitudinal bore of the first port, such that the specimen receptacle is pushed out of the longitudinal bore of the first port into a body cavity of a patient, and the inner shaft assembly is passed through the longitudinal bore of the second port, such that the support assembly exits the distal portion of the second port and enters the body cavity, to open a first opening of the specimen receptacle. A second opening of the specimen receptacle is removed from the body cavity, and a tissue specimen is passed through the first opening of the specimen receptacle, into the specimen receptacle. The tissue specimen is removed from the specimen receptacle, and the specimen retrieval device is removed from the body opening.

In embodiments, introducing the first port of the specimen retrieval device includes passing the distal portion of the first port through an incision. In other embodiments, introducing the first port of the specimen retrieval device includes passing the distal portion of the first port through a patient's vagina.

In some embodiments, passing the inner shaft assembly through the longitudinal bore of the second port includes passing resilient fingers of the support assembly which support the specimen receptacle through the longitudinal bore of the second port to allow the resilient fingers to move to a non-deformed state to open the first opening of the specimen receptacle.

In embodiments, passing the tissue specimen through the first opening of the specimen receptacle into the specimen receptacle includes utilizing a grasping device to grasp the tissue specimen. Suitable grasping devices include forceps, a tenaculum, and combinations thereof.

In other embodiments, methods of the present disclosure further include, prior to removing the tissue specimen from the specimen receptacle, introducing a third port through a second body opening; introducing a grasping device through the second port into the body cavity; grasping a mouth defining the second opening of the specimen receptacle with the grasping device; and removing the second opening of the specimen receptacle through the third port. In some embodiments, removing the second opening includes pulling the second mouth through the third port.

In embodiments, removing the tissue specimen from the specimen receptacle includes removing the tissue specimen through the second opening.

In other embodiments, removing the tissue specimen from the specimen receptacle includes removing the tissue specimen through the first opening.

In some embodiments, methods of the present disclosure include, prior to removing the tissue specimen from the specimen receptacle, withdrawing the support assembly from the body cavity through the longitudinal bores of the first and second ports to remove the first opening from the body cavity.

In embodiments, methods of the present disclosure include breaking up the tissue sample prior to removing the tissue specimen from the specimen receptacle.

### BRIEF DESCRIPTION OF THE DRAWINGS

Embodiments of the presently disclosed specimen retrieval device are described herein with reference to the drawings wherein:

FIG. 1 is a perspective view of an exemplary embodiment of a specimen retrieval device in accordance with the present disclosure prior to insertion of a second port of the specimen retrieval device into a first port of the specimen retrieval device and prior to deployment of a specimen receptacle;

FIG. 2 is an exploded view of the specimen retrieval device shown in FIG. 1;

FIG. 3 is a side, cross-sectional view of the specimen retrieval device shown in FIG. 1;

FIG. 4 is a side, cross-sectional view of the specimen retrieval device shown in FIG. 1 with the second port slightly inserted into the first port;

FIG. 5 is a cross-sectional view of the specimen retrieval device shown in FIG. 4 with the second port fully inserted into the first port and the specimen receptacle partially deployed;

FIG. 6 is a perspective view of the specimen retrieval device shown in FIG. 2, showing deployment of the specimen receptacle therefrom;

FIG. 7 is a side view showing the specimen retrieval device shown in FIG. 1 positioned in a patient's body cavity, showing a third port to be used in conjunction with the specimen retrieval device;

FIG. 8 is a side view of the specimen retrieval device shown in FIG. 7, showing passage of a free end of the specimen receptacle through the third port;

FIG. 9 is a side view of the specimen retrieval device shown in FIG. 8, showing placement of a tissue specimen into the specimen receptacle; and

FIG. 10 is a side view of the specimen retrieval device shown in FIG. 9, showing the tissue specimen in the specimen receptacle.

### DETAILED DESCRIPTION

The present disclosure provides a specimen retrieval device for use in minimally invasive surgical procedures. As used herein with reference to the present disclosure, minimally invasive surgical procedures encompass laparoscopic procedures and endoscopic procedures, and refer to procedures utilizing scopes or similar devices having relatively narrow operating portions capable of insertion through a small incision in the skin.

Aspects of the present disclosure may be modified for use with various methods for retrieving tissue specimens during minimally invasive surgical procedures, sometimes referred to herein as minimally invasive procedures. Examples of minimally invasive procedures include, for example, cholecystectomies, appendectomies, nephrectomies, colectomies, splenectomies, laparoscopic hysterectomies, and the like.

As used herein, the term “distal” refers to that portion of a specimen retrieval device which is farthest from the user, while the term “proximal” refers to that portion of the specimen retrieval device which is closest to the user.

The present disclosure provides a specimen retrieval device including two (2) ports for introducing a specimen receptacle into a patient's body. One port has a smaller diameter than the other port, permitting insertion of the smaller diameter port into the bore of the larger diameter port.

Embodiments of the present disclosure will now be described in detail with reference to the drawings, in which like reference numerals designate identical or corresponding elements in each of the several views. In the following description, well-known functions or constructions are not described in detail to avoid obscuring the present disclosure in unnecessary detail.

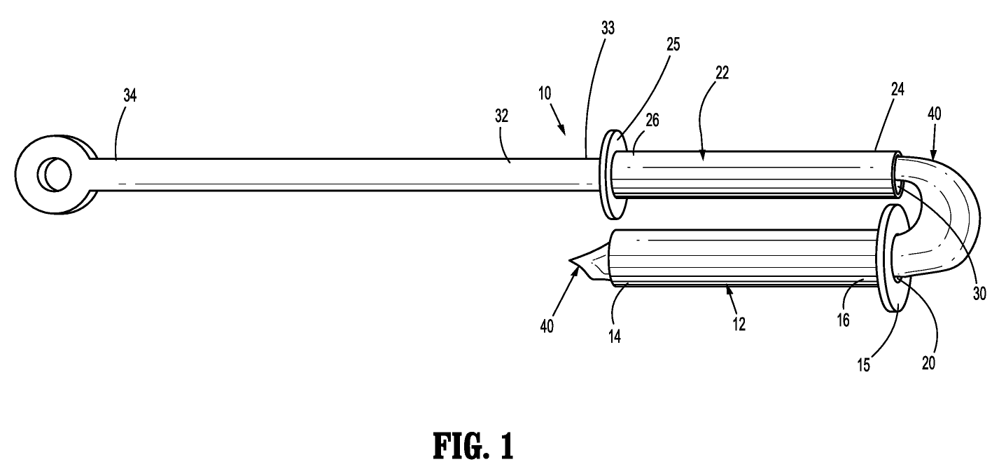

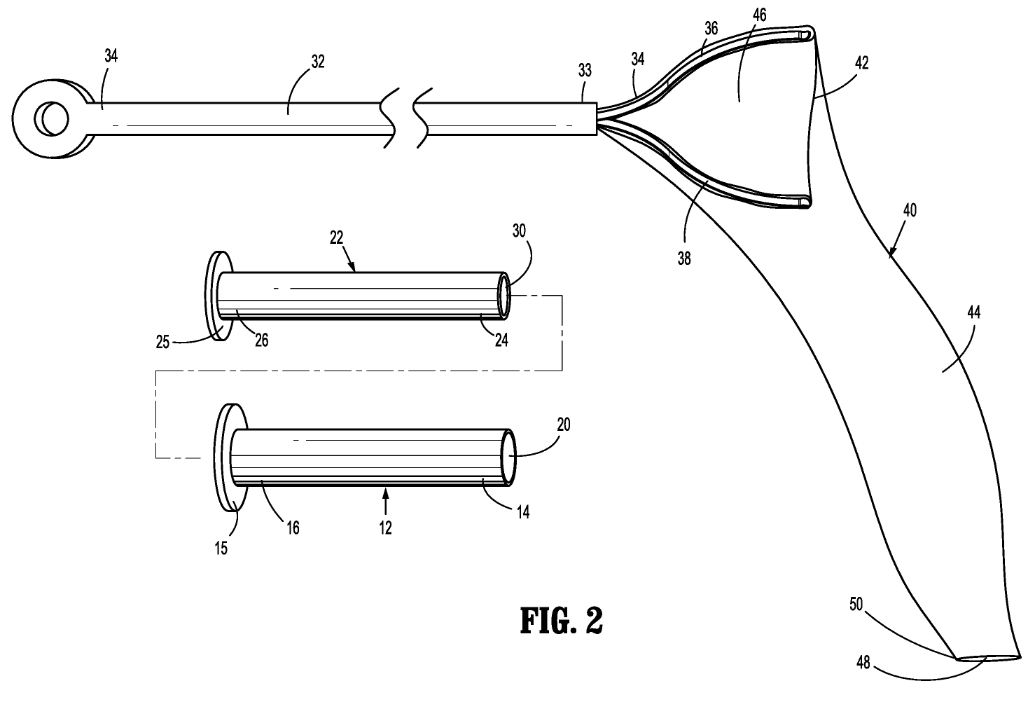

With reference to FIGS. 1-10, and initially with reference to FIGS. 1 and 2, a specimen retrieval device 10 according to an embodiment of the present disclosure is illustrated. The specimen retrieval device 10 includes a first port 12 defining a longitudinal bore 20 and including a distal portion 14 and a proximal portion 16. The specimen retrieval device 10 also includes a second port 22 defining a longitudinal bore 30 and including a distal portion 24 and a proximal portion 26. The second port 22 is of a smaller diameter than the first port 12, permitting insertion of the distal portion 24 of the second port 22 into the longitudinal bore 20 of the first port 12 and movement of the second port 22 within the longitudinal bore 20 of the first port 12 in telescopic fashion from the proximal portion 16 to the distal portion 14 of the first port 12.

The first port 12 may also include a flange 15 positioned about the proximal portion 16 and an end of the longitudinal bore 20. The flange 15 is configured to prevent the first port 12 from passing into a patient's body cavity. The second port 22 may similarly include a flange 25 positioned about the proximal portion 26 and an end of the longitudinal bore 30. The flange 25 is configured to prevent the second port 22 from passing completely into the longitudinal bore 20 of the first port 12.

In embodiments, the first port 12 and/or the second port 22 of the specimen retrieval device 10 of the present disclosure is made of biocompatible materials within the purview of those skilled in the art, in embodiments, polymeric materials. For example, thermoplastic polyurethanes sold under the name PELLETHANE®, offer flexibility and a wide range of hardness. The first port 12 and/or the second port 22, for example, may be fabricated from PELLETHANE® 2363-80A, PELLETHANE® 2363-90A, PELLETHANE® 2363-55D, any combination thereof, or any alternatives within the purview of those skilled in the art.

In embodiments, the specimen retrieval device 10 of the present disclosure includes an inner shaft 32 that is dimensioned to be slidably positioned within the longitudinal bore 20 of the first port 12 and/or the longitudinal bore 30 of the second port 22. The inner shaft 32 has a proximal portion 34, a distal portion 33, and a specimen receptacle 40 supported on the distal portion 33 of the inner shaft 32. As depicted in FIGS. 1 and 2, in embodiments the proximal portion 34 of the inner shaft 32 may have a gripping member such as a circular or ring configuration to enhance manipulation and deployment of the inner shaft 32.

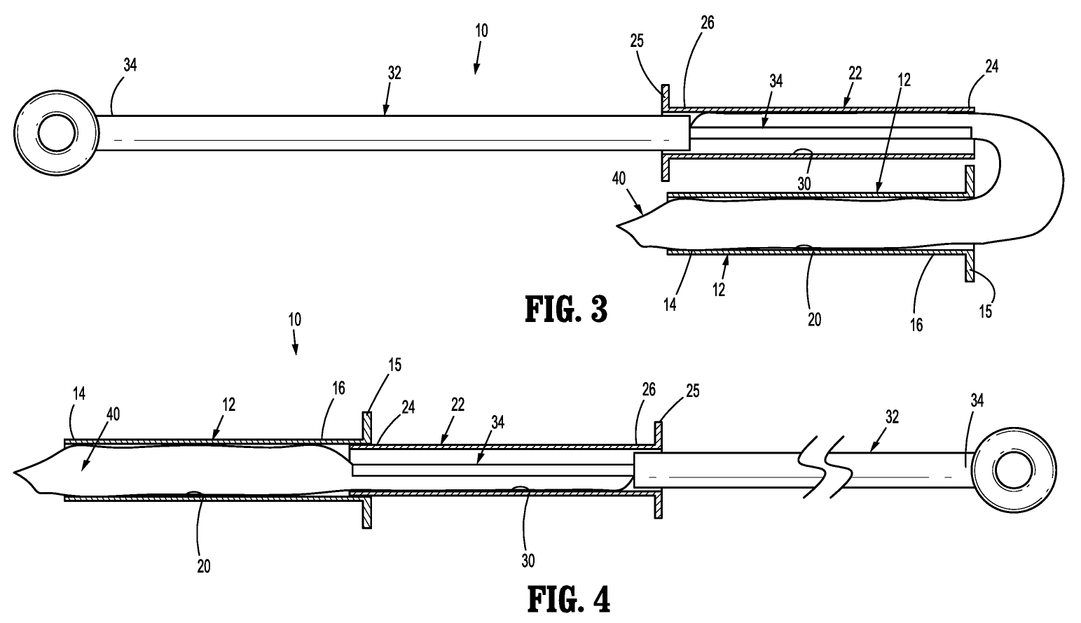

Referring to FIGS. 2 and 3, the specimen receptacle 40 includes a body 44 having a generally tubular or elongated configuration that includes an openable and closable portion 42 defining a first opening 46. The specimen receptacle 40 also has a mouth 50 defining a second opening 48 spaced at a distance from the first opening 46. Alternatively, other specimen receptacle configurations are envisioned. The distal portion 33 of the inner shaft 32 is coupled to a support member 34 that is configured to support the specimen receptacle 40. In embodiments, the support member 34 includes a pair of resilient fingers 36, 38 that extend distally from the distal portion 33 of the inner shaft 32. In embodiments, the resilient fingers 36, 38 can be integrally formed with the distal portion 33 of the inner shaft 22 such as by molding. Alternately, the specimen receptacle 40 can be supported on the distal portion 33 of the inner shaft 32 using other fastening or securing techniques.

The resilient fingers 36, 38 are movable from a spaced non-deformed state (FIG. 2) to a deformed state (FIGS. 3 and 4) to facilitate placement of the specimen receptacle 40 into the longitudinal bore 30 of the second port 22. The resilient fingers 36, 38 return to the non-deformed state when the specimen receptacle 40 is deployed from the longitudinal bore 30 of the second port 22 to open the opening 46 of the specimen receptacle 40, as described below.

The body 44 of the specimen receptacle 40 may be made from any suitable biocompatible material (e.g., nylon, urethane, ripstop nylon or latex) capable of forming a flexible collapsible member, or membrane. In embodiments, the material from which the specimen receptacle is made is resilient, antistatic, pyrogen-free, non-toxic, and sterilizable. In embodiments, materials used to form the first port 12, the second port 22, and/or the inner shaft 32 described above may be used to form the specimen receptacle 40. In other embodiments, the specimen receptacle 40 is formed of materials that are different from those used to form the first port 12, the second port 22, and/or the inner shaft 32. The specimen receptacle 40 may be opaque or clear.

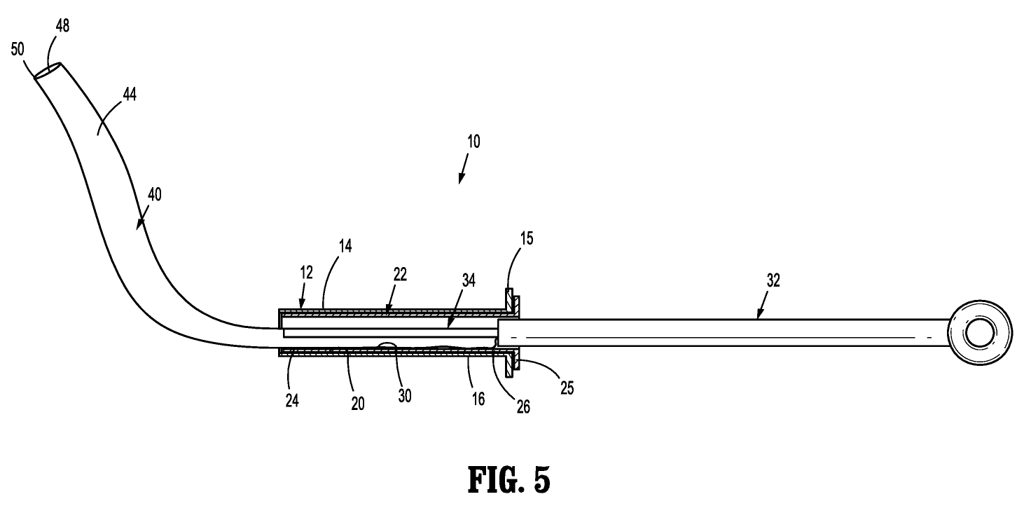

As depicted in FIGS. 4 and 5, the specimen receptacle 40 is placed within the bore 20 of the first port 12. The distal portion 24 of the second port 22 is then placed within the bore 20 of the first port 12 at the proximal portion 16 of the first port 12.

As shown in FIG. 5, the movement of the second port 22 within the bore 20 of the first port 12 ejects a portion of the specimen receptacle 40 from the distal portion of 14 the first port 12 so that the second opening 48 and the body 44 of the specimen receptacle 40 are positioned externally of the first port 12.

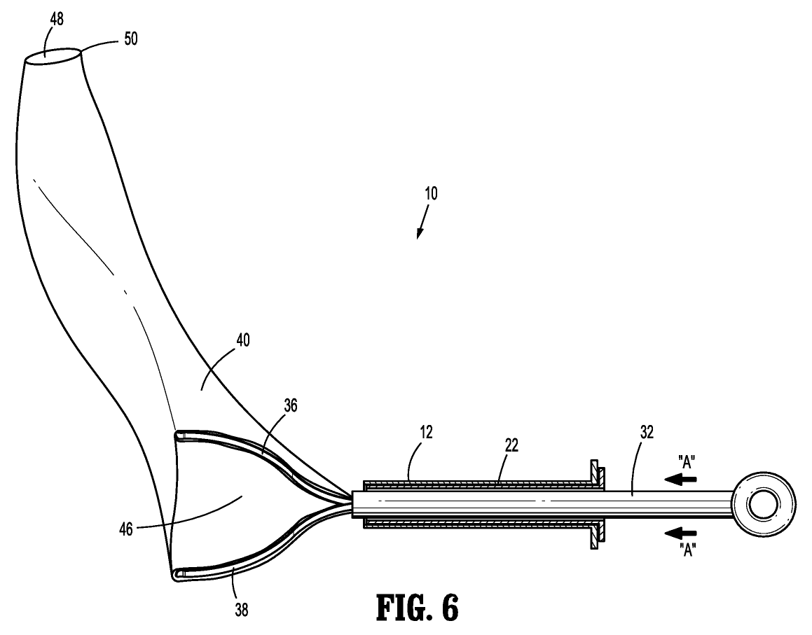

Turning to FIGS. 5 and 6, movement of the inner shaft 32 in a distal direction (indicated by arrows “A” in FIG. 6) through the bore 30 of the second port 22 deploys the resilient fingers 36, 38 from within the bore 30 of the second port 22 so that the resilient fingers 36, 38 return to their non-deformed state, forming first opening 46 (FIG. 6) of the specimen receptacle 40.

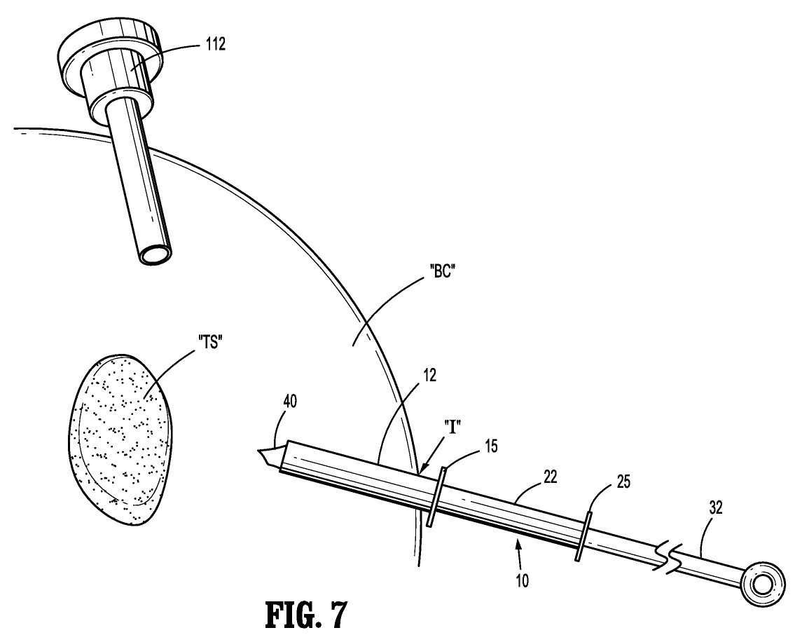

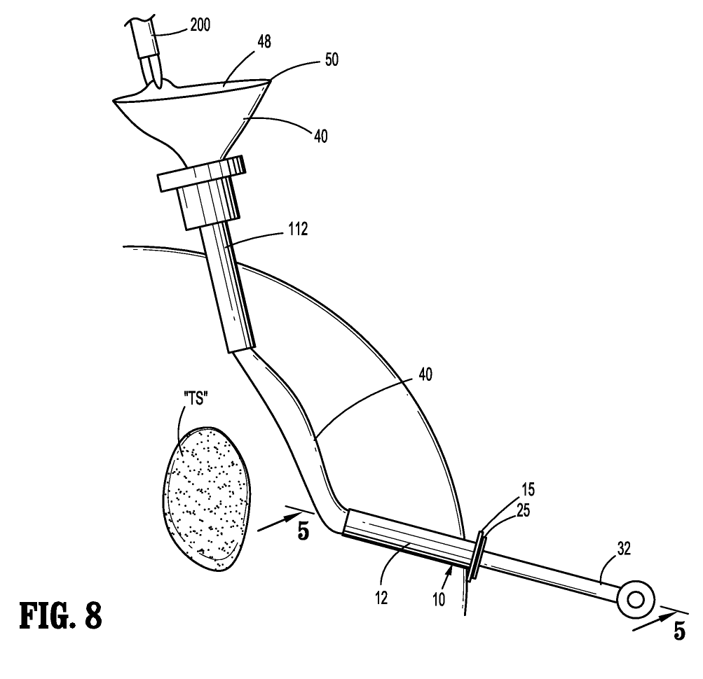

In use, as depicted in FIGS. 7-10, the specimen retrieval device 10 is inserted through an incision “I” into a patient's body cavity “BC”. This occurs by first introducing the distal portion 14 of the first port 12 of the specimen retrieval device 10 through the incision “I” (FIG. 7). The flange 15 on first port 12 prevents passage of the first port 12 through the incision “I” into the body cavity “BC”. After introduction of the first port 12 through the incision “I”, the second port 22 may be introduced into the bore 20 of the first port 12 (FIG. 8). Introduction of the second port 22 into the bore 20 of the first port 12 and pushing or advancing the second port 22 distally within the bore 20 of the first port 20 will assist in introducing the specimen receptacle 40 into the body cavity “BC”. The flange 25 on the second port 22 prevents the second port 22 from passing entirely into the bore 20 of the first port 12. The inner shaft 32 may then be pushed distally through the bore 30 of the second port 22 so that resilient fingers 36, 38 are introduced into the body cavity “BC”, at which point the resilient fingers 36, 38 open to their non-deformed state, thereby forming the first opening 46 of the specimen receptacle 40.

Kits of the present disclosure may include both the specimen retrieval device described herein, as well as grasping devices such as a forceps or a tenaculum, trocars, cannulas, scalpels, vacuum sources (tubes), inflation sources, additional ports, combinations thereof, and the like.

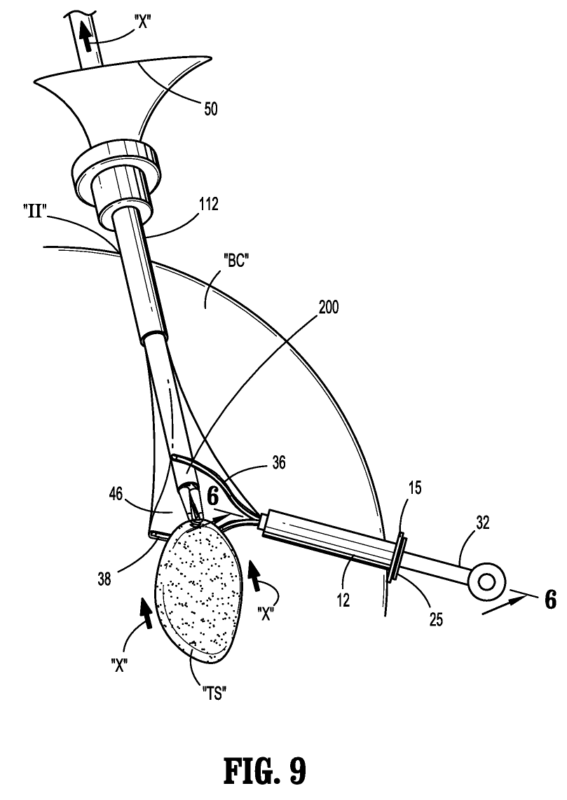

With specific reference to FIG. 9, in embodiments, a kit of the present disclosure includes a third port 112 placed through a second incision “II” and a grasping device 200 (e.g., a tenaculum, forceps, etc.) configured to pass through the third port 112. The grasping device 200 may be used to pull the mouth 50 of the specimen receptacle 40 defining the second opening 48 out of the body cavity “BC” through the third port 112. After the mouth 50 is pulled through the third port 112, the grasping device 200 must be inserted through the mouth 50 and through the specimen receptacle 40 to grasp a tissue specimen “TS”. Proximal pulling of the grasping device 200 (indicated by arrows “X” in FIG. 9), facilitates proximal pulling of a tissue specimen “TS” into the specimen receptacle 40 through the first opening 46, through the body 44, and out the mouth 50 defining the second opening 48 of the specimen receptacle 40.

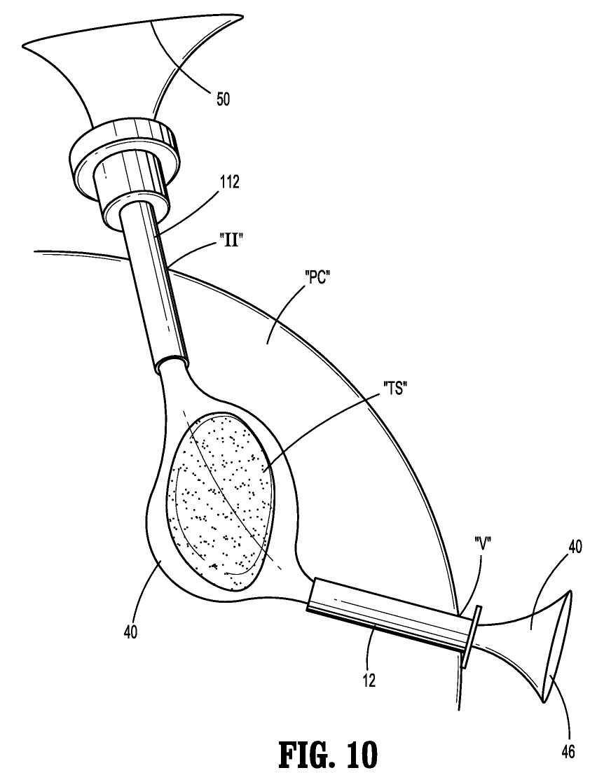

Alternatively, as depicted in FIG. 10, in embodiments the closable portion 42 defining the first opening 46 of the specimen receptacle 40 may also be removed from the body cavity “BC” through the specimen retrieval device 10. For example, the second port 22 and inner shaft 32 of the specimen retrieval device 10 may be pulled proximally (not shown) out of the body cavity “BC” such that the resilient fingers 36, 38 move to the deformed state, out of the body cavity “BC” and through the bore 20 of the first port 12 so that the first opening 46 is externalized. The tissue sample “TS” may then be removed through the first opening 46 of the specimen receptacle 40. In some embodiments, the closable portion 42 of the first opening 46 may be optionally attached to the first port 12 (not shown) and/or the mouth 50 defining the second opening 48 may be optionally attached to the third port 112 (not shown), to prevent the first opening 46 and the second opening 48 from being pulled back into the body cavity “BC” (not shown).

Where the tissue sample “TS” is too large to remove the tissue sample “TS” through the second port 22 and/or the third port 112, a tissue extraction device, tenaculum, (not shown) or similar device may be introduced through first opening 46 or the second opening 48 to break up tissue sample “TS” within the specimen receptacle 40 prior to its removal from the specimen receptacle 40 (not shown). Examples of such procedures, including the use of a tenaculum and radiofrequency (RF) energy to break up the tissue specimen “TS” to aid in its removal from the specimen receptacle 40 are disclosed in U.S. Patent Application Publication No. 20160058495, the entire disclosure of which is incorporated by reference herein.

Alternatively, the specimen retrieval device 10 of the present disclosure may be used for transvaginal procedures. For example, devices of the present disclosure may be used during a laparoscopic hysterectomy or a similar procedure where vaginal access is part of the procedure. FIG. 10 shows first port 12 inserted through a patient's vagina “V”. Introduction of the specimen retrieval device 10 occurs as outlined above, with the exception that the first port 12 is introduced through the vagina, instead of a first incision. The rest of the procedure is the same.

While previous specimen receptacles and/or specimen bags may be utilized to remove smaller tissue samples, the dual openings on the specimen receptacles of the present disclosure permit the construction of larger specimen receptacles in combination with ports that remain small to minimize trauma to a patient upon placement in an incision.

In embodiments, the specimen receptacles of the present disclosure may be useful for the removal of large tissue specimens from a body cavity. For example, the specimen retrieval devices of the present disclosure may be used to extract large tissue specimens during a total laparoscopic hysterectomy. A trans-vaginal approach allows for the use of a larger specimen receptacle and a morcellation device to access the abdominal cavity without the need for a larger abdominal incision. Due to the large size of the specimen receptacle, it is packed in the two separate tubular ports. To minimize the length of the product in the packaging, the tubes are not connected at first (allowing them to be folded side-by-side). When removed from the packaging, they will be connected and then inserted into the vagina.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto. For example, while the above disclosure describes in detail two separate ports that collapse into each other, it is envisioned that the ports could be manufactured so that they are connected to each other with a first port sliding within the bore of a second port in a telescopic fashion.

Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Accordingly, the invention is not to be limited by what has been particularly shown and described, except as indicated by the appended claims.

What is claimed is:

1. A specimen retrieval device, comprising:
a first port defining a first longitudinal bore and having a proximal portion and a distal portion, the first longitudinal bore having a first diameter;
a second port defining a second longitudinal bore and having a proximal portion and a distal portion, the second longitudinal bore having a second diameter smaller than the first diameter;
an inner shaft assembly for passage through the first and second longitudinal bores, the inner shaft assembly having a proximal portion and a distal portion, the distal portion of the inner shaft assembly having a support assembly affixed thereto; and
a specimen receptacle supported on the support assembly, the specimen receptacle including a body defining a first opening that may be opened and closed and a second opening spaced from the first opening.

2. The specimen retrieval device of claim 1, wherein the proximal portion of the first port includes a flange, the flange being dimensioned to prevent passage of the first port into a patient's body cavity.

3. The specimen retrieval device of claim 1, wherein the proximal portion of the second port includes a flange, the flange being dimensioned to prevent passage of the second port completely into the first longitudinal bore.

4. The specimen retrieval device of claim 1, wherein the support assembly includes a pair of resilient fingers which support the specimen receptacle, the resilient fingers being movable from a deformed position within the longitudinal bore of the second port to a non-deformed state to open a first mouth of the specimen receptacle to form the first opening in a deployed state.

5. A kit comprising:
the specimen retrieval device of claim 1; and
at least one additional component including cannulas, grasping devices, or combinations thereof.

6. A method comprising:
introducing a first port of a specimen retrieval device through a body opening, the first port defining a longitudinal bore of a first diameter and having a proximal portion and a distal portion;
introducing a second port into the longitudinal bore of the first port, the second port defining a longitudinal bore of a second diameter and having a proximal portion and a distal portion, the second diameter being smaller than the first diameter;
introducing an inner shaft assembly into the longitudinal bores of the first and second ports to position a specimen receptacle supported on a support assembly on the distal portion of the inner shaft within the longitudinal bores of the first and second ports, the inner shaft assembly having a proximal portion and a distal portion;
passing the second port distally through the longitudinal bore of the first port, such that the specimen receptacle is pushed out of the longitudinal bore of the first port into a body cavity of a patient;
passing the inner shaft assembly through the longitudinal bore of the second port, such that the support assembly exits the distal portion of the second port and enters the body cavity, to open a first opening of the specimen receptacle;
removing a second opening of the specimen receptacle from the body cavity;
passing a tissue specimen through the first opening of the specimen receptacle, into the specimen receptacle;
removing the tissue specimen from the specimen receptacle; and
removing the specimen retrieval device from the body opening.

7. The method of claim 6, wherein introducing the first port of the specimen retrieval device includes passing the distal portion of the first port through an incision.

8. The method of claim 6, wherein introducing the first port of the specimen retrieval device includes passing the distal portion of the first port through a patient's vagina.

9. The method of claim 6, wherein passing the inner shaft assembly through the longitudinal bore of the second port includes passing resilient fingers of the support assembly which support the specimen receptacle through the longitudinal bore of the second port to allow the resilient fingers to move to a non-deformed state to open the first opening of the specimen receptacle.

10. The method of claim 6, wherein passing the tissue specimen through the first opening of the specimen receptacle into the specimen receptacle includes utilizing a grasping device to grasp the tissue specimen.

11. The method of claim 10, wherein the grasping device is selected from the group consisting of a forceps, a tenaculum, and combinations thereof.

12. The method of claim 6, further including, prior to removing the tissue specimen from the specimen receptacle:
introducing a third port through a second body opening;
introducing a grasping device through the second port into the body cavity;
grasping a mouth defining the second opening of the specimen receptacle with the grasping device; and
removing the second opening of the specimen receptacle through the third port.

13. The method of claim 12, wherein the grasping device includes a forceps, a tenaculum, and combinations thereof.

14. The method of claim 12, wherein removing the second opening includes pulling the second mouth through the third port.

15. The method of claim 12, wherein removing the tissue specimen from the specimen receptacle includes removing the tissue specimen through the second opening.

16. The method of claim 12, further including, prior to removing the tissue specimen from the specimen receptacle, withdrawing the support assembly from the body cavity through the longitudinal bores of the first and second ports to remove the first opening from the body cavity.

17. The method of claim 12, wherein removing the tissue specimen from the specimen receptacle includes removing the tissue specimen through the first opening.

18. The method of claim 12, further comprising breaking up the tissue sample prior to removing the tissue specimen from the specimen receptacle.
